(19) United States
(12) Patent Application Publication
(10) Pub. No.: US 2025/0001444 A1
(43) Pub. Date: Jan. 2, 2025

(54) DELIVERY DEVICE WITH A GAS SENSOR AND METHOD FOR THE OPERATION THEREOF

(71) Applicant: SCHEUGENPFLUG GMBH, Neustadt (DE)

(72) Inventors: Sebastian Piller, Nüremberg (DE); Stefan Dollinger, Hemhofen (DE)

(73) Assignee: SCHEUGENPFLUG GMBH, Neustadt (DE)

(21) Appl. No.: 18/576,062

(22) PCT Filed: May 20, 2022
(86) PCT No.: PCT/EP2022/063722

§ 371 Date: Aug. 30, 2024

(30) Foreign Application Priority Data
Jul. 2, 2021 (DE) 10 2021 117 163.6

(51) Int. Cl. B05B 12/00; B05B 15/25; G01N 21/64; G01N 27/404; G01N 33/00

(52) U.S. Cl. CPC B05B 12/004; B05B 15/25; G01N 21/64; G01N 27/404; G01N 33/0027

(57) ABSTRACT

A discharge device for discharging a material comprising a liquid or paste, in particular self-leveling material. The device includes a consumer for discharging the material, a preparation unit having a preparation container for preparing the material, or a conveying unit for conveying the material from a storage container to the consumer. A gas sensor is used for determining gas content of a sample gas in the material. In some cases, the sample gas is a measuring component of a gas mixture to be measured in the material. In some examples, the material may not contain any gas at all. The amount of sample gas is determined as close as possible to the consumption location by means of a Clark sensor or an optical sensor. In the case of air, for example, the oxygen content is determined, and the total content of air in the material is calculated therefrom.

### I. APPLICATION AREA

The invention relates to a discharge device for liquid and viscous material, such as adhesives, casting resins or casting compounds, in the preparation unit of which this material is not only stored, but is also prevented from demixing by stirring and circulating, and especially is also degassed. This is because, for further use in a consumer-typically a discharge unit-gas, in particular air, in particular visible air inclusions, or even dissolved air, are extremely disadvantageous in the material.

In this context, the meaning of dissolved air is in dispute. The majority of the opinions define dissolved air as a small accumulation of air in the form of very small air bubbles that are no longer visible in the material. This is not to be confused with ions of the chemical elements of which air consists.

### II. TECHNICAL BACKGROUND

Such a preparation unit first comprises the preparation container, which usually has a round inner cross-section in plan view, so that when a stirrer rotating about an upright axis is arranged therein, the stirring blade thereof can usually be moved along the circumference close to the inner surfaces of the wall and/or the floor of the storage container.

For the material to be prepared, there is a stationary, generally non-central, inflow opening in the preparation container in its upper region, and an outflow opening in the lower region, which is usually coupled to one or two conveying pumps in order to convey the material to the consumer via a supply line.

The material is usually degassed via thin-film degassing, which involves the material which flows into the preparation container via the inflow opening not being supplied directly to the filling process for the storage container, but rather flowing onto a dissipator, the upper side of which—the distribution surface—is above the maximum filling level, serving to distribute the flow of the material, wherein a layer of the material that is as thin as possible can be formed on this 30 distribution surface, from which any remaining air inclusions open up more easily the thinner the layer is. This is because the thinner the layer is, the greater the probability that an air inclusion reaches the upper side of the layer and thus opens, and the contained gas, usually air, is released to the surroundings.

In addition, a very low pressure of usually below 20 mbar, often below 5 mbar, often below 2 mbar, prevails in the air space of the preparation container in order to facilitate the outgassing of air inclusions from the thin layer. Other degassing methods can also be applied.

The material should not have any gas—usually air—not even in dissolved, non-visible form, and in particular no visible gas inclusions, since even such small gas fractions negatively impact the product produced from the material—for example the electrical properties of the cast product, for example the dielectric resistance of a potting compound around an electrical circuit.

A distinction is made between visible and invisible gas inclusions, wherein “dissolved gases” is the terms used to describe the non-visible gas inclusions. Even in the technical literature, it is disputed whether these are microscopically small bubbles or actually physically/chemically dissolved gases, i.e., gas ions.

For example, water, whether it is in a lake in the open air or in an aquarium, has a certain oxygen content which limits bioactivity, and which fish can breathe.

The problem is that the gas content in such a viscous, preferably self-leveling material has not been measured until now, and thus the quality and quantity of the degassing cannot be verified and is not reproducible.

There are, however, sensors which can measure certain types of gas in a liquid to determine the content.

So-called Clark sensors are amperometric sensors and can measure the oxygen content in a liquid. They are sold, for example, by Mettler-Toledo and usually used in pharmacies, food technology, or in the brewing sector, because the oxygen content is frequently of interest there.

However, amperometric sensors consume a portion of the gas, in particular oxygen, which they measure during the measurement.

However, there are also optical sensors for sample gas content in a liquid, in which this consumption is lower or zero.

These optical sensors are usually based on the principle that an emitted, defined electromagnetic radiation is partially absorbed by the sample gas in the material, and thus the difference between the emitted radiation quantity and reflected radiation quantity is a measure of the gas content in the material.

Frequently, a layer which is a component of the sensor, rather than the material itself, is irradiated, and the reflective properties thereof are changed by the sample gas with which it is brought into contact.

### III. Explanation Of The Invention

a) Technical Object

It is therefore the object according to the invention to provide a discharge device and a method for determining the gas content in a liquid or paste, in particular for determining that a pre-specified maximum permissible gas content of the material is reached, or not reached, in particular during the degassing thereof, with which the determination of the gas content is possible even in the case of self-leveling but high-viscosity materials, even at a gas content below 5% by volume, even below 3% by volume or even below 2% by volume in the material.

b) Solution to the Object

This object is achieved by the features of claims 1 and 12. Advantageous embodiments result from the subclaims.

With regard to the method, the object is achieved in that the material is held in contact with a gas mixture or is in any event in contact with a gas mixture, the composition of which is known and can only change slightly, before the measurement, i.e., in particular during the production, storage and preparation.

This offers the possibility of measuring only the content of one of the components of the gas mixture in the material, the measuring component, so that it is possible to select the most easily measurable component-or a component which can be measured at all, in contrast to the other components of the gas mixture.

The total content of gas, namely of the entire gas mixture, in the material can then be deduced from the content of this measuring component.

The inventive step is therefore to select such a gas mixture whose composition is known and from which the content of at least one component can be measured in the material.

In practice, the gas in the surrounding environment of the material is almost always air, and the oxygen content of this air is almost always the same worldwide—assuming that it has not been altered by specific influencing factors, such as an elevated consumption by, for example, local combustion processes.

A gas mixture which contains oxygen is thus preferably used, since its content in the material can be measured. Preferably, therefore, air is usually selected as a gas mixture, assuming that it can be selected at all.

Preferably either an amperometric measuring method which consumes the measuring component during the measurement, or an optical measuring method is used as the measuring method, both of which measure even very small, no longer visible, air inclusions, i.e., so-called dissolved air.

In an amperometric measuring method, an electrolysis current is usually measured between two electrodes, which is directly proportional to the concentration of the substance converted at the one electrode, in a substance mixture of the measuring component.

In a so-called Clark sensor, which likewise contains two electrodes, usually one made of silver and one of platinum, contained in a single rod-shaped sensor—but confusingly often referred to as a “Clark electrode” overall—dissolved oxygen is reduced at a constant electrical potential. The electrodes are generally separated from the material by a membrane which is permeable to oxygen.

Chrono-amperometry and bi-amperometry also fall under the amperometric measuring methods.

In an optical measuring method for quantitative determination of gas in a liquid, electromagnetic radiation of such a wavelength toward the material to be evaluated/measured is preferably directed against the material, which electromagnetic radiation is able to excite a boundary layer which is optically active between the radiation source and the material at the given frequency. The excitation emits light of a defined emission spectrum, and is detected by a suitable photosensitive sensor.

The presence of oxygen and/or of the gas component to be measured reduces the intensity of the light emitted by the excitation, and/or its emission spectrum.

Consequently, the light emitted by the optically active boundary layer of the sensor correlates with the gas content of the material in contact with it.

This is also referred to as fluorescence quenching in cases where an optically active, in particular fluorescent, layer is used as a reflective layer.

In the case of a measuring method consuming the sample gas, in particular the measuring component, such as amperometry, the measurement is preferably carried out only when it can be assumed that the same content thereof is present between the electrodes as in the remaining material, in particular of the total remaining material on average.

This can be ensured by a sufficient flow of material in the region of the electrodes.

This also applies in the case of optical sensors in which a measurement should only be carried out if, at the boundary layer which reflects the radiated electromagnetic radiation, the same content of the sample gas, in particular the measuring component, is present as in the remaining material-for example by means of sufficient flow.

In this case, it must be ensured, in the case of a consuming measuring method, that the flow along the sensor is sufficiently great for the quantity of the sample gas or the measuring component continuously supplied by the flow to be greater than the amount consumed in the same time unit by the measuring method.

A reduced pressure of below 100 mbar, in particular below 50 mbar, in particular below 20 mbar, in particular below 10 mbar, often prevails in the surrounding environment of the material with the gas. However, this only has to be taken into account for providing the necessary flow.

The corresponding sensor in this case usually has to be in contact with the material—preferably even in the case of an optical sensor—since the electromagnetic radiation cannot be directed directly onto the surface of the material and reflected there. Rather, it is directed onto an optically active layer whose optical properties change by contact with the sample gas. This is why this optical layer must be in contact with the material in which the sample gas is present.

Furthermore, a so-called optical insulation, for instance black silicone, should be present on the material side of the optically active layer, which is usually arranged at the front end of the sensor, in order to prevent electromagnetic radiation other than the radiation radiated by the optical sensor from striking the optical sensor element.

In a consuming measuring method, such as amperometry, the sensor element is preferably covered by the material by a membrane permeable to the sample gas, wherein the diffusion capability of the membrane for the sample gas should of course be as high as possible so that the ratio of the sample gas content on the two sides of the membrane is as close to 1 as possible.

Preferably, the two contents should be in a known ratio to one another or, for example, ascertainable by calibration, which is taken into account in the calculation of the gas content.

Usually, the material is classified as ready-to-use with regard to the gas content, preferably automatically, by means of the controller, which is connected for signal exchange to the at least one gas sensor, if a permissible upper threshold of the gas content is not reached.

In particular, the material is also supplied to the consumer only if, and/or when, preparation in a preparation container is carried out upstream thereof at a preparation location, then the removal from the preparation container is carried out only if the material located therein is classified as ready-to-use with regard to the gas content.

The preparation container is usually refilled with material in batches or continuously from a storage container.

The material is preferably classified as ready-to-use with regard to the gas content only if
a pre-specified upper threshold of the gas content was not exceeded over a pre-specified continuity period,
and/or
the decrease in the gas content per unit of time during a decrease period was lower than a pre-specified decrease threshold.

Only then can the sample gas contents be considered to be sufficiently stable.

Furthermore, a continuous measurement of the gas content would result in an enormous amount of data which would need to be prepared. In addition, in particular in the case of aggressive, in particular abrasive, material and constant flow of this material to the sensor, the contact surface would wear quickly.

Therefore, especially in the case of aggressive, in particular abrasive, material,
the sensor is only exposed to the, in particular flowing, material during the measurement periods,
in particular outside the measurement periods, the material is routed past the sensor.

Furthermore, it is advantageous to know the behavior of the material being monitored as well as possible, with regard to the gas content, most of all during degassing.

Therefore, for a new material, in order to learn the degassing behavior of the new material, new material is preferably degassed as a teach-in degassing, from the same starting state of the gas content over a degassing time, and the gas content is measured, preferably under different degassing scenarios in the form of degassing parameters influencing the degassing, in particular the ambient pressure and/or the material temperature during the degassing and/or the quantity of energy introduced during the degassing and/or for the movement of the material.

Preferably, for each teach-in degassing, the minimum achievable gas content is determined or estimated-in particular, the overall minimum achievable gas content which is expected given an optimal combination of degassing parameters, which need not have been present in any of the teach-in degassings. This can take place on the basis of the different degassing behaviors in the different degassing scenarios and their degassing parameters.

In this way, in particular with the knowledge of, or after determination of the degassing behavior of, the material, the optimal degassing parameters, for example the ambient pressure during degassing, and/or the optimal operating parameters of the consumer, for example the throughput of the consumer, can in particular be determined automatically, and the material can accordingly be prepared particularly quickly or particularly economically into a ready-to-use state.

The optimal parameters are defined in particular as a function of pre-specified, preferably staggered, optimization targets-for example, the energy consumption during the preparation of the material and/or the required minimum throughput of the consumer and/or an optimally short preparation time for the material from an initial state to the ready-to-use state.

Especially for this purpose, the gas content is measured during the degassing—but in particular only at time intervals—in order to keep the costs, in particular of the data evaluation, in a reasonable ratio to the gains in process reliability achieved as a result.

Preferably, the gas content in the discharge device is measured not only at one point, but rather at two points spaced apart in the direction of flow of the material. Two approaches are possible, and can also be used in combination with one another:

Either the measurement is carried out at two points where the gas content should be the same, or a certain difference or relation to one another should exist between them—for example, at the outlet of a preparation container on the one hand and at the input of the consumer supplied therefrom on the other hand-so that the gas contents measured in this case can be regarded as redundant measurement values, thereby revealing for example, that the sensors are functioning correctly, or the state of the discharge device between the two measuring points is correct.

And/or the measurement is carried out at two such points where the gas content should be different-for example, at the material inlet of a preparation container on the one hand, and at the material outlet on the other hand-so that the difference reveals, for example, properties of the preparation device therebetween, in particular the degree of degassing therebetween, or leaks therebetween.

With regard to the location of the measurement of the gas content, the content should be measured as close as possible to the consumption location, in particular at the consumer, since the gas content in the spring accumulator consumption of the material is the essential information.

If the material is prepared according to the requirements of a consumer and then is consumed by a consumer, and in particular is discharged, at a consumption location removed from the preparation location,
the content is measured at the preparation location
and/or
the content is measured in a circulation line at the preparation location,
and/or
the content is measured in a conveying unit conveying the material, such as a pump.

A discharge device for liquid or paste material can comprise different modules, typically
a discharge unit as a consumer and/or
a preparation unit with a preparation container for processing the material according to the requirements of a downstream consumer
and/or
a supply container with material, in particular the original container supplied by the manufacturer of the material,
and/or
a conveyor unit for conveying the material to the consumer, whether from a storage container
and/or
connecting lines between the modules.

This function is fulfilled by the preparation unit because the discharge device has at least one gas sensor for quantitative determination of the content of a sample gas in the material, in particular of the measuring component of a gas mixture, in particular even in dissolved form.

The measurement is then preferably carried out as close as possible to the consumption location, since a maximum permissible gas content must be reliably maintained precisely there.

Alternatively, the measurement can be carried out in a supply line from the preparation location to the consumption location. This offers the advantage that the preferred flow is provided there at the gas sensor—at least periodically.

The same applies to the measurement in a circulation line at the preparation location, by means of which the stored material is recirculated at the preparation location in a preparation container.

Measurements made directly in such a preparation container should be performed at a point at which there is sufficient flow, for example by means of a movement device present in the preparation container, such as an agitator, or in the outlet of the preparation container.

The gas sensor is then positioned at the location at which, at least periodically, a sufficient flow prevails, in order to use the continued delivery to compensate for the consumption of sample gas close to the sensor.

In general, in the case of a gas sensor that consumes the sample gas, said gas sensor should be arranged at a position in the discharge device where there is a flow of the material along the contact surface of the sensor where the content of sample gas is measured or through which sample gas enters the sensor, at least during the operation of the discharge device, in order to prevent the formation of a stationary boundary layer of material, or a boundary layer of material that moves very little along the contact surface, wherein the gas content thereof would then not be representative of the gas content remote from the boundary layer.

This can be achieved, for example, with an oblique position of the contact surface relative to the flow direction, such that the flow flows onto the contact surface, in particular if the gas sensor and thus its contact surface is arranged in a pipeline or hose line for the material.

Preferably, the preparation container, which can be tightly sealed, has a vacuum connection in its upper region, i.e., above the level of material, so that the pressure is kept below 900 mbar, preferably below 500 mbar, preferably below 100 mbar, preferably below 50 mbar, preferably below 20 mbar, preferably below 10 mbar, preferably below 5 mbar, in the air space above the material and thus in the entire preparation container, to facilitate the opening of air inclusions in the material.

Preferably, a removal of the material from the preparation container for delivery to the consumer is only permitted if the permissible maximum gas content is not reached.

If the gas potentially present in the material is air, a sensor measuring the content of oxygen is preferably used as a gas sensor, because the content of air in total can be calculated in the material therefrom.

Preferably, the gas sensor is used in a position contacting the material. This is required in some sensor types anyway.

Preferably, an amperometric gas sensor, in particular a Clark sensor, is used, since such sensors are available as a purchased part.

Such a consuming gas sensor is preferably separated from the material by a membrane which is permeable to the sample gas.

The diffusion capability of the membrane for the sample gas must be great enough that a substantially constant gradient of the content of the sample gas between the two sides of the membrane is established, which should be as close to 1 as possible.

Alternatively, the contents on both sides of the membrane are in a known ratio to one another, and can be taken into account when determining the total content of gas in the material.

Alternatively, an optical sensor is used, the emitted radiation of which is reflected by the gas fraction, in particular its measuring component, in the material.

An optical insulation is preferably provided between the optical sensor and the material so that only electromagnetic radiation emitted by the optical sensor arrives at its sensor element in reflected form-and no other light.

c) Embodiments

Embodiments in accordance with the invention are described in more detail below by way of example. In the figures:

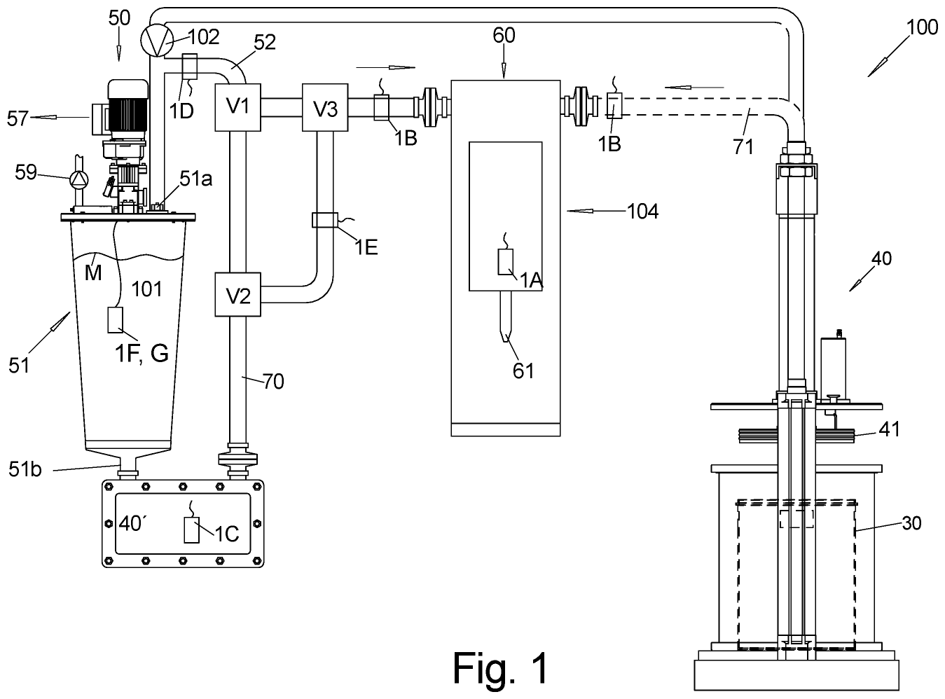

FIG. 1: is an example of a discharge device, with possible positions for the attachment of a gas sensor,

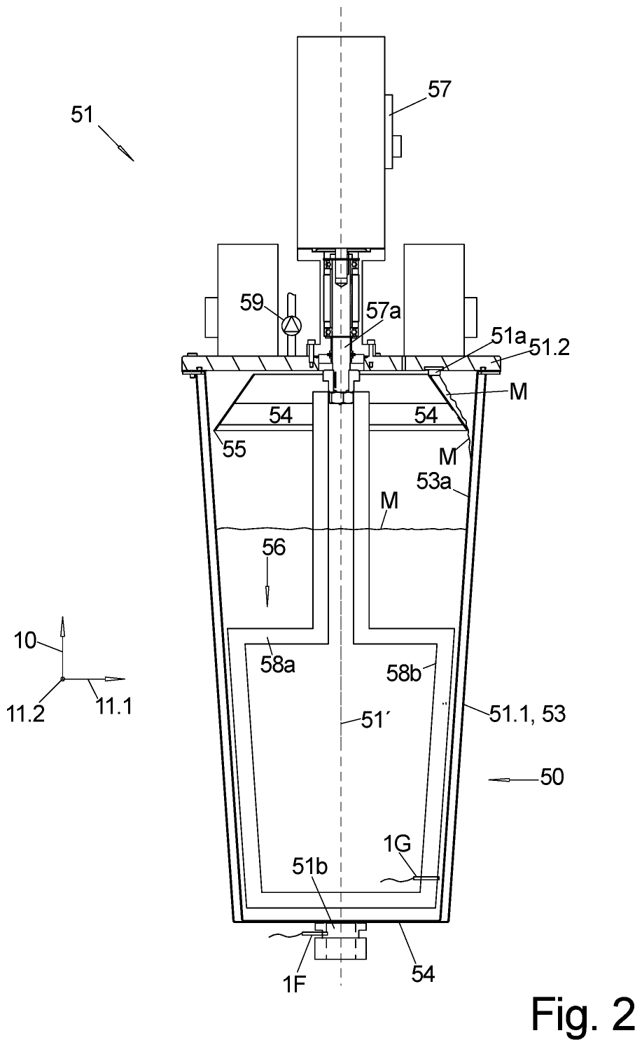

FIG. 2: is a vertical section through a preparation container,

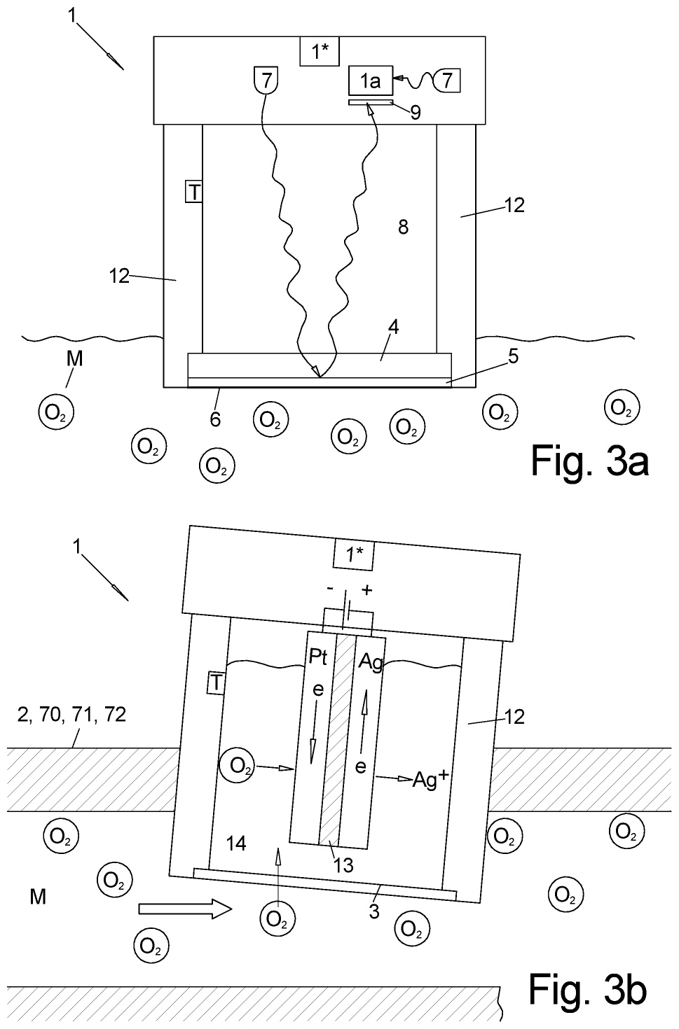

FIG. 3a: is a functional diagram for an optical sensor,

FIG. 3b: is a function diagram for an amperometric sensor.

FIG. 1 shows a discharge device 100 with a discharge unit 60 as a consumer of the discharge device 100, wherein material is dispensed in a metered manner to a component (not shown) via a discharge nozzle 61.

On the left and right connection sides of the discharge unit 60—in this form, hypothetically—the following can be present, more in alternating arrangement than simultaneously:

The right connection side shows how material M is removed from a storage container 30 for the material M by means of a conveying unit 40 directly, i.e., without preparation, and is supplied to the discharge unit 60 via a connecting line 71, in this case by means of a selection sequence emptying device 40 with a selection sequence plate 41, wherein the storage container 30 is the original container, a so-called hobbock, in which the material is supplied by the manufacturer.

The left connection side shows the far more frequent case that the consumer 60 is supplied with prepared material M from a preparation unit 50—which is refilled from the supply container 30.

There, the material M is prepared, for example mixed and degassed, in a preparation container 51, and from there is supplied to the consumer 60 via a conveying unit 40′ usually in the form of a pump and a connection line 70.

For the degassing, the preparation container 51 has a vacuum connection which is connected to a vacuum source 59. The material M is introduced via an inflow opening 51a and removed therefrom via an outflow opening 51b. The inflow to the inflow opening 51a usually takes place from the storage container 30 as shown in the right half of the figure.

In addition, a circulation line 52 can be present, via which material removed at the outflow opening 51b is again re-introduced via the inflow opening 51a in the upper region into the preparation container 51, by means of the pump 40′—in this case, the same pump 40 which also performs the transport of the material via the connection line 70 to the consumer 60. The circulation line 52 is partially identical to the connecting line 70, and the supply of the material M to the consumer 60 or to the inflow opening 51a can be controlled by means of a valve V1—preferably automatically.

FIG. 1 shows at which points within such a discharge device 100 a gas sensor 1 could be arranged—which gas sensor is usually accommodated in a sensor receptacle 2, which can, for example, have a flow opening and can be inserted into a pipeline, as shown in FIG. 3.

In principle, the aim is to measure the gas content as close as possible to the discharge point, i.e., the discharge nozzle 61, since the gas content is decisive there.

Preferably, therefore, the gas sensor 1 is arranged directly in the discharge unit 60, i.e., in the position A, preferably in its internal piping or flow channels, as close as possible upstream of the discharge nozzle 61, and thus the static or dynamic mixer tube-which, in the case of a material M consisting of a plurality of components, is mostly not shown and fastened thereto.

If this is not possible or not desired, such a gas sensor 1 could be arranged in the connecting line 70 or 71, i.e., in position B—then preferably as close as possible to the connection of the discharge unit 60.

However, the gas sensor 1 can also be arranged in the circulation line 52, preferably downstream of the valve V1, as position D.

Since the pump 40′ is generally located upstream of the valve V1 and usually directly connected, i.e., without upstream branching, to the outlet opening 51b of the preparation container 51, the gas sensor 1 can also be arranged as position C in the pump 40′, for example in its inlet socket or outlet socket.

Especially if aggressive, for example abrasive, materials M will be processed, it may also be useful to provide a bypass line 72 to the connection line 70 from the processing unit 50 to the consumer 60, wherein a switchable valve V2 is provided at least at the branch from the connecting line 70, and preferably also at its opening back into the connecting line 70.

A gas sensor 1 can then be provided as position E in the bypass 72, and the flow of material is routed via the gas sensor 1 there only at the times of the measurement, by means of corresponding switching of the valve V2, and optionally also V3. Outside of the measurement periods, the gas sensor is therefore not worn down by the abrasive material M.

If the gas sensor 1 is to be accommodated elsewhere, preferably the preparation unit 50, and preferably the preparation container 51, may be considered for this purpose.

Such a preparation container 51 is shown in FIG. 2 in cross section, and is made of a pot 51.1 open at the top and a lid 51.2 which tightly seals said pot on the upper side thereof.

In the interior of this preparation container 51, a stirrer 56 rotates with—in this case—two stirring blades 58a, b arranged on opposite sides with respect to the axis of rotation, which stirring blades 58a, b, in this case are fastened to a motor shaft 57a by their upper ends in a rotationally fixed manner, and extend downward from there close to the vertical axis of symmetry 51′ of the typically rotationally symmetrical container 51 as viewed in plan view, and also extend further outward in the lower region and run close to the inner surface of the wall 53 of the pot 51.1 when they are driven in a rotating manner.

The motor shaft 57a is the drive shaft of a motor 57, which represents the stirrer drive, and is arranged on the upper side of the cover 51.2, so that the motor shaft 57a passes through it from the top to the bottom.

An outflow opening 51b is shown centrally in the base on the axis of symmetry 51′.

A gas sensor 1 is shown at position F in the outflow opening 51b and/or the socket thereof directly in the outlet below the base, which would then measure the gas content in the material directly at the outlet, which would have the advantage that a flow of material M flows along the contact surface of the gas sensor 1.

For the same reason, a gas sensor 1 can be fastened as position G to one of the stirring blades 58b, preferably with its contact surface radially outward or inward, projecting beyond the stirring blade 58b as low as possible above the base, in order to be able to measure the gas content in the material M even at a low filling level in the preparation container 51.

A disadvantage then is that a power supply and a signal connection must be ensured from the rotating stirring blade to the controller.

Apart from the vacuum connection to the vacuum source 59, in this case one, but sometimes also two, inflow openings 51a are provided in the cover 51.2, for example in order to introduce two separate components into the preparation container 51 and mix them therein, wherein each inflow is controlled via a dedicated inlet valve seated thereabove.

The material M first runs out of the inflow opening 51a on the upper 20) side of a truncated-cone-shaped dissipator 54, which preferably likewise rotates—for example, rotates together with the stirrer 56, and there forms a thin layer which flows over its lower peripheral edge as a drip edge 55.

Since the pot 51.1 widens conically upwards and the drip edge 55 is located close to the inner surface 53a of the peripheral wall 53 of the pot 51.1, the 25 thin layer then runs further downward from the drip edge 55 on the inner surface of the peripheral wall, and the formation of these thin layers promotes the degassing of the material in addition to the vacuum prevailing in the preparation container 51.

FIG. 3a, b show the functional principles of two possible gas sensors 1:

FIG. 3a is an illustration of the principle of an optical gas sensor 1 for oxygen measurement:

The core element is an optically active layer 5, which changes its optical properties when it comes into contact with the sample gas—in this case, oxygen.

For example, the optically active layer 5 can be a reflective or even fluorescent layer whose reflective properties or fluorescence properties change all the more significantly as the oxygen per unit of time coming into contact with this optically active layer 5 increases.

For this purpose, the optical layer 5 is illuminated from one side by an exciting electromagnetic radiation, here visible light, from an LED 7, preferably guided through an optical fiber 8 or a strand made of glass fibers 8, and the light that is then reflected by the optical layer 5 and sent back in the same direction, e.g., fluorescent light is detected by a sensor element 1a. In this case, the reflected light arriving at the sensor element 1a can be filtered by means of an optical filter 9 for the frequency or frequency range emitted by the exciting LED.

Preferably, the sensor element 1a is also irradiated by a reference LED 7 which corresponds to the exciting LED 7 and emits light in the same quantity and with the same frequency or frequency range, so that the sensor element 1a, when measuring the light received from the reference LED 7 and the light received from the optically active layer 5—whether with regard to wavelength or quantity—relays the measurement results to the connected controller 1*, which converts the difference into a value representing the oxygen content in the material which is in contact with the optically active layer 5 during the measurement.

In order that no extraneous light can reach the optically active layer 5 from the side opposite the excitation side, it is coated with an optically insulating layer 6 (or has itself optically insulating properties) on the side facing away from the optically active layer, the material side, which, however, is permeable to the oxygen molecules.

In terms of design, the optically active layer 5 is mostly made of glass supported by a support plate 4 completely permeable to the exciting reflected light, wherein the optically insulating layer 6 is located on the material facing away from the support plate 4.

The circumference of this layer arrangement is placed tightly on or in the front end region of a guide tube 12 which also guides and peripherally protects the optical glass fiber 8 or the glass fiber strand 8.

The electrically operated components, i.e., the two LEDs 7, the sensor element 1a with the upstream optical filter 9 and the controller 1*, are accommodated at the other end of the guide tube 12, or even in the guide gate 12 in the other end region.

In addition, a temperature sensor T, positioned preferably on or in the inner circumference of the support tube 12, can be present in order to monitor the operating temperature of the optical sensor, since the measurement result is temperature-dependent and must possibly also be calibrated with regard to the temperature during the measuring process.

FIG. 3b is an illustration of the principle of a Clark sensor, i.e., an amperometric gas sensor 1, for oxygen measurement:

In this case, an anode, usually consisting of silver, and a cathode, usually consisting of platinum, which are electrically insulated from one another by means of an insulator 13, protrude into a generally liquid electrolyte 14 in which oxygen molecules are located. The anode and cathode are connected to one another via a voltage source, the operating voltage of which changes in proportion to the chemical reaction running in the sensor 1, namely:

At the cathode: O₂+4e⁻+2 H₂O=4 OH

At the anode: 4 Ag+4 Cl=4 Ag Cl+4e⁻

The connected controller 1* measures the change occurring in the operating voltage, and converts it into a value representative of the oxygen content in the material M.

In terms of design, the anode and the cathode, as well as the electrolyte 14, are located in a closed support tube 12, in the front end region of which a membrane 3, which sealingly closes its end face and is permeable to the oxygen in the material, is introduced, and the rear end of which is closed, is flowed through only at the rear ends of the electrodes or the electrical supply lines thereof.

In FIG. 3b, the sensor 1 is installed in a manner creating a seal with its axial direction, i.e., the running direction of its support tube 12, transverse but not precisely perpendicular in the wall of a sensor receptacle 2 or a line 70, 71, 72, in an inclined position so that the contact surface formed by the outside of the membrane 3 faces the flow of material M prevailing at the location of the sensor 1 in the line or the sensor receptacle 2 in particular with an inclination of a few degrees.

A temperature sensor T can also be present here, preferably in the inner circumference of the support tube 2, which monitors the operating temperature of the gas sensor 1 and reports to the controller 1*, so that the gas sensor 1 can then be recalibrated or the representative value can be adapted to the measured temperature.

### List of Reference Numbers

1, 1.1, 1.2 Gas sensor
1* Controller
1a, b Sensor element
2 Sensor receptacle
3 Membrane
4 Support plate, glass plate
5 Optically active layer
6 Optically insulating layer
7 LED
8 Glass fiber, glass fiber bundle
9 Optical filter
10 Verticals
11.1, 11.2 Horizontal transverse direction
12 Support tube
30 Storage container
40 Conveying unit, selection sequence emptying device
40 Conveying unit, pump
41 Selection sequence plate
50 Preparation unit
51 Preparation container
51.1 Pot
51.2 Lid
51 Axis of symmetry, center, axis of rotation
51a Inflow opening
51b Outflow opening
52 Circulation line
53 Wall
53a Inner surface
54 Dissipator
55 Edge, drip edge
56 Stirrer
57 Stirrer drive, motor
57a Motor shaft, drive shaft
58a, b, c Stirrer blade
59 Vacuum source
60 Consumer, discharge unit
61 Discharge nozzle
70 Connecting line
71 Connecting line
72 Bypass line
100 Discharge device
100* Controller
A-G Position
1-26. (canceled)

27. A discharge device for discharging a material comprising a liquid or paste, comprising:
at least one of a consumer for discharging the material, a preparation unit having a preparation container for preparing the material, or a conveying unit for conveying the material from a storage container to the consumer; and
at least one gas sensor for determining a gas content of a sample gas in the material, wherein the sample gas is a measuring component of a gas mixture to be measured in the material.

28. The discharge device according to claim 27, wherein the gas sensor is arranged in the preparation unit, in at least one of the following positions:
on a stirrer blade in the preparation unit,
in a circulation line on the preparation unit,
in or on the consumer,
in or on the conveying unit, or
in connection lines between the conveying unit and a discharge unit.

29. The discharge device according to claim 27, wherein the gas sensor is an oxygen-measuring gas sensor.

30. The discharge device according to claim 27, wherein the gas sensor is in contact with the material.

31. The discharge device according to claim 27, wherein the gas sensor is a sensor that consumes the measuring component of the gas mixture.

32. The discharge device according to claim 27, wherein the gas sensor is an optical sensor, and the optical sensor comprises an optically active layer between a radiation source and the material;
wherein the material is excited by incident radiation emitted from the radiation source and emits secondary radiation of a defined emission spectrum;
wherein the secondary radiation is detected by a suitable sensor; and
wherein the presence of the sample gas in the material changes at least one of the intensity or the emission spectrum of light emitted by excitation of the material.

33. The discharge device according to claim 27, wherein the gas sensor is an amperometric sensor.

34. The discharge device according to claim 33, wherein sensor elements on the amperometric sensor are covered by a membrane which is permeable to the sample gas.

35. The discharge device according to claim 27, wherein the gas sensor is a Clark sensor for measuring oxygen content in a liquid.

36. The discharge device according to claim 27, wherein the gas sensor is arranged at a position in the discharge device where there is a flow of the material along a contact surface of the gas sensor;
wherein the content of the sample gas is measured at the contact surface or the sample gas enters into the gas sensor at least during the operation of the discharge device.

37. The discharge device according to claim 36, wherein the gas sensor is arranged in a pipeline or hose line for the material.

38. The discharge device according to claim 36, wherein the gas sensor is an amperometric sensor;
wherein a movement device is present in the preparation container to maintain the flow of the material to the contact surface of the gas sensor; and
wherein the flow of the material is strong enough such that a rate of continuous delivery of the sample gas that takes place as a result of the flow of the material to the contact surface of the gas sensor is greater than the consumption of the sample gas by the gas sensor.

39. The discharge device according to claim 27, wherein an upper region of the interior of the preparation container or an upper region of the storage container can be brought into connection with a vacuum source; and
wherein the vacuum source is able to keep the gas mixture in a gas chamber of the preparation container above the material at a pressure below 900 mbar.

40. A method for determining a gas content in a liquid or paste material in a discharge device, the discharge device including at least one of a consumer for discharging the material, a preparation unit having a preparation container for preparing the material, or a conveying unit for conveying the material from a storage container to the consumer, the method comprising:
using at least one gas sensor for determining a gas content of a sample gas in the material, wherein the sample gas is a measuring component of a gas mixture to be measured in the material, wherein the composition of the gas mixture is known; and
determining a total content of the gas mixture from the gas content of the measuring component by extrapolation.

41. The method according to claim 40, wherein the gas mixture contains oxygen.

42. The method according to claim 40, wherein the gas sensor is an optical sensor for measuring oxygen content in the material or wherein the gas sensor is an amperometric sensor.

43. The method according to claim 40, wherein a sensor principle is applied in which the sample gas is consumed at least partially by the gas sensor; and
wherein a measurement is carried out if it can be assumed that the gas content of the sample gas is the same at a boundary layer of the material as in a remainder of the material.

44. The method according to claim 40, further comprising:
connecting an upper region of the interior of a preparation container of the discharge device or an upper region of a supply container of the discharge device to a vacuum source; and
maintaining the pressure of the gas mixture below 900 mbar.

45. The method according to claim 40, placing the gas sensor into contact with the material in a manner that prevents the material from forming a slow-moving or stationary boundary layer of the material relative to the sensor.

46. The method according to claim 40, further comprising, maintaining a sufficiently strong flow of the material at a boundary layer on the gas sensor during measurement, such that continued delivery of the sample gas to a contact surface of the gas sensor is greater than a rate of consumption of the sample gas by the gas sensor.

47. The method according to claim 40, wherein the gas sensor is an amperometric sensor and sensor elements on the amperometric sensor are covered by a membrane which is permeable to the sample gas, wherein the diffusion capability of the membrane for the sample gas is known; and
wherein the method further comprises determining a ratio of the gas content of the sample gas on both sides of the membrane.

48. The method according to claim 40, wherein the gas content is measured in or at at least one of: the consumer, the preparation container, a circulation line at the preparation location, or a conveying unit.

49. The method according to claim 40, further comprising classifying the material as ready-to-use with regard to the gas content if a permissible upper threshold of the gas content is not reached.

50. The method according to claim 49, further comprising:
refilling the preparation container with the material from a storage container; and
permitting a supply of the material from the preparation container to the consumer if the material is classified as ready-to-use with respect to the gas content.

51. The method according to claim 40, further comprising classifying the material as ready-to-use with regard to the gas content if a pre-specified upper threshold of the gas content is not exceeded over a pre-specified continuity period and/or a decrease in the gas content during a decrease period is below a pre-specified decrease threshold.

52. The method according to claim 40, wherein the material is an abrasive material and wherein the method further comprises:
exposing the gas sensor to the material during measurement periods; and
outside the measurement periods, routing the material past the gas sensor.
